(19) United States
(12) Patent Application Publication
(10) Pub. No.: US 2008/0287543 A1
(43) Pub. Date: Nov. 20, 2008

(54) Pharmaceutical composition comprising 5-methyl-2 (2'-chloro-6'-fluoroanillino) phen ylacetic acid

(76) Inventors: Rose-Marie Dannenfelser, Bloomfield, NJ (US); Vivia Christine Georgousis, West caldwell, NJ (US); Maha Y. Khaled, Livingston, NJ (US); Tarun S. Patel, Parsippany, NJ (US); Joseph Sikora, Saccasunna, NJ (US); Barbara Wang, Berkeley Heights, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/220,060

(22) Filed: Jul. 21, 2008

Related U.S. Application Data
(63) Continuation of application No. 10/574,537, filed on Aug. 11, 2006.
(60) Provisional application No. 60/509,459, filed on Oct. 8, 2003.

(51) Int. Cl. A61K 31/195; A61P 29/00

(52) U.S. Cl. 514/567

(57) ABSTRACT

The invention relates to a composition for the treatment of a cyclooxygenase-2-mediated disorder or condition comprising 5-methyl-2-(2′-chloro-6′-fluoroanilino)phenylacetic acid or a pharmaceutically acceptable salt, preferably the potassium salt, thereof suitable for parenteral administration, and to a method for the treatment of a cyclooxygenase-2-mediated disorder or condition in a human or animal in need of such treatment by parenteral administration of 5-methyl-2-(2′-chloro-6′-fluroanilino)phenylacetic acid or a pharmaceutically acceptable salt, preferably the potassium salt, thereof.

The present invention relates to a composition for the treatment of a cyclooxygenase-2-mediated disorder or condition comprising 5-methyl-2-(2′-chloro-6′-fluoroanilino)phenylacetic acid or a pharmaceutically acceptable salt thereof suitable for parenteral administration, and to a method for the treatment of a cyclooxygenase-2-mediated disorder or condition by parenteral administration of 5-methyl-2-(2′-chloro-6′-fluoroanilino)phenylacetic acid or a pharmaceutically acceptable salt thereof.

In a preferred embodiment, the present invention relates to a composition for the treatment of a cyclooxygenase-2-mediated disorder or condition in a human or animal in need of such treatment, the composition comprising a liquid suitable for parenteral administration of 5-methyl-2-(2′-chloro-6′-fluoroanilino)phenylacetic acid or a pharmaceutically acceptable salt thereof.

In another preferred embodiment, the present invention relates to a method for the treatment of a cyclooxygenase-2-mediated disorder or condition in a human or animal in need of such treatment, the method comprising administering an effective amount of a composition of the invention, i.e. of a composition comprising a liquid suitable for parenteral administration of 5-methyl-2-(2′-chloro-6′-fluoroanilino)phenylacetic acid or a pharmaceutically acceptable salt thereof.

All patents, patent applications, and other publications referred to herein are hereby expressly incorporated by reference in their entirety. In case of a conflict between the present specification and material incorporated by reference, the present specification is controlling.

The utility of 5-methyl-2-(2′-chloro-6′-fluoroanilino)phenylacetic acid, in free form or in pharmaceutically acceptable salt form, and methods for its synthesis are disclosed in U.S. Pat. No. 6,291,523, according to which disclosure a genus of compounds, including 5-methyl-2-(2′-chloro-6′-fluoroanilino)phenylacetic acid, is useful for, inter alia, the relief of pain, fever and inflammation associated with a variety of disorders or conditions including rheumatic fever, symptoms associated with influenza or other viral infections, common cold, low back and neck pain, dysmenorrhea, headache, including migraine headache, toothache, sprains and strains, myositis, neuralgia, synovitis, arthritis, including osteoarthritis and rheumatoid arthritis, degenerative joint diseases, gout and ankylosing spondylitis, bursitis, bums, and injuries following surgical and dental procedures. It is desirable to provide a liquid parenteral dosage formulation comprising 5-methyl-2-(2′-chloro-6′-fluoroanilino)phenylacetic acid or a pharmaceutically acceptable salt thereof for the treatment of a human or animal suffering from any of the aforementioned disorders or conditions, e.g. from acute pain.

It has now surprisingly been found, that a shelf-stable liquid parenteral dosage formulation comprising a pharmaceutically acceptable salt, especially the potassium salt, of 5-methyl-2-(2′-chloro-6′-fluoroanilino)phenylacetic acid can be prepared. 5-methyl-2-(2′-chloro6′-fluoroanilino)phenylacetic acid, i.e. the free acid, is relatively insoluble in water, and it also degrades in water. Thus, the ability to produce a shelf-stable parenteral formulation is unexpected. Furthermore, 5-methyl-2-(2′-chloro-6′-fluoroanilino)phenylacetic acid, i.e. the free acid, is quite unstable in polyethylene glycol (PEG) 400 (showing about 19% degradation in a solution of 100% PEG 400 at 50° C. in the dark after 4 weeks, compared with only 5% degradation of the potassium salt of 5-methyl-2-(2′-chloro-6′-fluoroanilino)phenylacetic acid under the same conditions) and in propylene glycol (PG) (showing about 71% degradation in a solution of 100% PG at 50° C. in the dark after 4 weeks, compared with only 7% degradation of the potassium salt under the same conditions). Solutions comprising 5-methyl-2-(2′-chloro-6′-fluoroanilino)phenylacetic acid or a pharmaceutically acceptable salt thereof can also be quite irritating upon injection or infusion, thus, the preparation of a liquid formulation suitable for parenteral administration is further unexpected.

The liquid parenteral dosage formulation of the invention comprises, in the form of an aqueous suspension or preferably an aqueous solution, a pharmaceutically acceptable salt of 5-methyl-2-(2′-chloro-6′-fluoroanilino)phenylacetic acid, particularly the potassium salt, as drug substance. The concentration of the drug substance may be between about 10 and about 80 mg of the free acid/ml, typically between about 10 and about 60 mg of the free acid/ml, preferably between about 20 and about 50 mg of the free acid/ml, more preferably between about 30 and about 40 mg of the free acid/ml, most preferably about 40 mg of the free acid/ml, the equivalent amount of the potassium salt of 5-methyl-2-(2′-chloro-6′-fluoroanilino)phenylacetic acid being, in each case, about 1.13 times as much.

The formulation of the invention typically also contains a cosolvent for the pharmaceutically acceptable salt, especially the potassium salt, of 5-methyl-2-(2′-chloro-6′-fluoroanilino)phenylacetic acid, such as propylene glycol, polyethylene glycol 400 or glycerin. In general, such a cosolvent is present in an amount of between about 5 and about 50%, preferably of between about 20 and about 50%, more preferably of between about 25 and about 45%, especially of between about 30 and about 45%, more especially of between about 35 and about 45%, most preferably of about 40%, by weight.

The formulation of the invention typically also contains a surfactant, e.g. a polysorbate, such as polyoxyethylene (20) sorbitan monooleate (polysorbate 80), a polyoxypropylene-polyoxyethylene block copolymer, such as Pluronic F-68 (having a molecular mass of about 7500), or a polyethoxylated castor oil, such as a Cremophor. Such a surfactant is typically present in an amount of between about 0.1% and about 10%, preferably of between about 0.5% and about 5%, more preferably of between about 1% and about 5%, especially of about 1% or of about 2% or of about 3% or of about 4% or of about 5%, by weight.

The formulation of the invention may also contain an antioxidant, such as ascorbic acid, a tocopherol, sodium sulfite, sodium metabisulfite, glutathione, thiourea, L-cysteine hydrochloride monohydrate, N-acetylcysteine or a monothioglycerol. Depending upon the antioxidant used, an antioxidant may typically be present in an amount of between about 0.01% and about 4%, preferably of between about 0.05% and about 3%, more preferably of between about 1% and about 2%, most preferably of about 2%, by weight.

The drug substance is most stable at a pH value of the parenteral formulation of between about 8.5 and about 10.5. Formulations with a pH value lower than about 8.5 contain relatively high levels of a cyclic degradation product, while those with a pH value higher than about 10.5 contain increased levels of an oxidative degradation product. Therefore, the formulation of the invention may also contain a buffer. Suitable buffers are e.g. glycine buffers or phosphate buffers.

The formulation of the invention can be prepared e.g. by admixing their components with water until a suspension or preferably a clear solution is obtained. The suspension or preferably the clear solution may be purged with nitrogen or another inert gas, e.g. argon, in order to minimize the amount of dissolved oxygen, which can increase the degradation of the drug substance. Nitrogen or another inert gas may be layered over the liquid in the containner for the formulation. Glass containers, such as vials or ampoules, are preferred. Clear glass containers are most preferred, although any suitable container, that is consistent with parenteral administration, can be used. As the drug substance is sensitive to light, it is also useful to further package formulations that are inside clear glass containers into further light opaque packaging, such as cardboard boxes. These methods for the preparation of the formulation of the invention are further embodiments of the present invention.

In another embodiment, the present invention relates to the use of a pharmaceutically acceptable salt, especially of the potassium salt, of 5methyl-2-(2′-chloro-6′-fluoroanilino)-phenylacetic acid for the preparation of a pharmaceutical composition for the treatment of a cyclooxygenase-2-mediated disorder or condition.

The example which follows is intended to illustrate the invention and does not limit the invention.

### EXAMPLE

### Solution for Parenteral Administration

| Ingredient | Amount | |
|---|---|---|
| Potassium salt of 5-methyl-2- | 45.2 | mg |
| (2' -chloro-6' - | | |
| fluoroanilino)phenylacetic acid | | |
| Polyethylene glycol 400 | 400 | mg |
| Polysorbate 80 | 20 | mg |
| Monothioglycerol | 2.0 | mg |
| Glycine | 7.5 | mg |

| | |
|---|---|
| Water purified, USP | q.s. to 1 ml |
| Sodium hydroxide, USP/NF | q.s. to pH 9.0 |

The ingredients are mixed, and the mixture is purged with nitrogen. As soon as a clear solution is obtained, it is transferred to a clear glass ampoule, and nitrogen is layered on top of the solution, after which the ampoule is sealed.

1. A composition which comprises a pharmaceutically acceptable salt of 5-methyl-2-(2′-chloro-6′-fluoroanilino)phenylacetic acid, a cosolvent, and surfactant.

2. A composition according to claim 1, which further comprises water.

3. A composition according to claim 2, which is in the form of a solution.

4. A composition according to claim 3, in which the cosolvent is a member, selected from the group, consisting of propylene glycol, polyethylene glycol 400 and glycerin.

5. A composition according to claim 3, in which the surfactant is a member, selected from the group, consisting of a polysorbate, a polyoxypropylene-polyoxyethylene block copolymer and a polyethoxylated castor oil.

6. A composition according to claim 3, which further comprises an antioxidant.

7. A composition according to claim 6, in which the antioxidant is a member, selected from the group, consisting of ascorbic acid, a tocopherol, sodium sulfite, sodium metabisulfite, glutathione, thiourea, L-cysteine hydrochloride monohydrate, N-acetylcysteine and a monothioglycerol.

8. A composition according to claim 6, which further comprises a buffer.

9. A composition according to claim 8, in which the buffer is a member, selected from the group, consisting of a glycine buffer and a phosphate buffer.

10. A composition according to claim 8, in which the pharmaceutically acceptable salt of 5-methyl-2-(2′-chloro-6′-fluoroanilino)phenylacetic acid is the potassium salt.

11. A composition according to claim 8, in which the cosolvent is polyethylene glycol 400.

12. A composition according to claim 11, in which the surfactant is a polysorbate.

13. A composition according to claim 12, in which the antioxidant is a monothioglycerol.

14. A composition according to claim 13, in which the buffer is a glycine buffer.

15. A composition according to claim 14, which further comprises a glass container, selected from the group, consisting of a vial and an ampoule.

16. A composition according to claim 15, characterized in that the solution is disposed in the glass container.

17. A method for minimizing the chemical degradation of the potassium salt of 5-methyl-2-(2′-chloro-6′-fluoroanilino)phenylacetic acid in an aqueous solution comprising the said salt, which method comprises adjusting the pH value of the aqueous solution to between about 8.5 and about 10.5.

18. A method for increasing the local tolerance while parenterally administering a composition comprising the potassium salt of 5-methyl-2-(2′-chloro-6′-flouroanilino)phenylacetic acid, which method comprises administering the said salt in the form of an aqueous solution that also comprises a cosolvent.

19. A method according to claim 18, characterized in that the cosolvent is polyethylene glycol 400.

20. A method for the treatment of a cyclooxygenase-2-mediated disorder or condition, which comprises parenterally administering a composition according to claim 1.

21. A method according to claim 20 for the treatment of a cyclooxyqenase-2-mediated disorder or condition, which comprises parenterally administering a composition according to claim 14.
